(19) United States
(12) Patent Application Publication
(10) Pub. No.: US 2015/0272551 A1
(43) Pub. Date: Oct. 1, 2015

(54) Module for Processing Ultrasonic Signal Based on Spatial Coherence and Method for Processing Ultrasonic Signal

(71) Applicants: Waygence Co., Ltd., Gangwon-do (KR); Industry Academic Cooperation Foundation, Hallym University, Gangwon-do (KR); Daejin University Center for Educational Industrial Cooperation, Gyeonggi-do (KR)

(72) Inventors: Mok-Geun Jung, Seoul (KR); Baek-Sub Kim, Gangwon-do (KR)

(21) Appl. No.: 14/265,641

(22) Filed: Apr. 30, 2014

(30) Foreign Application Priority Data
Mar. 26, 2014 (KR) 10-2014-0035009

(51) Int. Cl. A61B 8/08

(52) U.S. Cl. CPC A61B 8/5223; A61B 8/5207

(57) ABSTRACT

Disclosed are a module for processing an ultrasonic signal based on spatial coherence and a method for processing an ultrasonic signal, and more particularly, to a module for processing an ultrasonic signal based on spatial coherence and a method for processing an ultrasonic signal that is configured to obtain an enhanced ultrasonic image by processing a reflected ultrasonic signal based on characteristics of a reflector or a medium. A module for processing an ultrasonic signal receives an ultrasonic signal reflected from the inside of a human body and forms an image based on the ultrasonic signal.

### CROSS-REFERENCE TO RELATED APPLICATIONS

This application claims priority under 35 USC 119(a) to Korean Application No. 10-2014-0035009 filed on Mar. 26, 2014, the disclosure of the prior application being incorporated herein in its entirety by reference in the disclosure of this application.

### BACKGROUND

1. Field

Apparatuses and methods consistent with the exemplary embodiments relate to a module for processing an ultrasonic signal based on spatial coherence and a method for processing an ultrasonic signal, and more particularly, to a module for processing an ultrasonic signal based on spatial coherence and a method for processing an ultrasonic signal that is configured to obtain an enhanced ultrasonic image by processing a reflected ultrasonic signal based on characteristics of a reflector or a medium.

2. Description of the Related Art

Various types of diagnosis methods and treatment methods using characteristics of ultrasound that is transmitted to a human body as a medium are publicly known. As a representative example of medical ultrasonography, an ultrasound-based diagnostic imaging technique refers to a technique that is used to detect and visualize a distance between an ultrasonic transducer and a boundary surface of a medium within internal body and echo signals for diagnosis. The ultrasound-based diagnostic imaging technique may be classified into A-mode diagnostic imaging technique, M-mode diagnostic imaging technology, B-mode diagnostic imaging technique and Doppler mode (D-mode) diagnostic imaging technology.

As one of the ultrasound-based diagnostic imaging technique, the B-mode has been developed focusing on a beam-forming method for forming a proper ultrasonic beam for human body using a plurality of ultrasonic array elements and on a signal processing technology. In the B-mode, signals that have been transmitted by respective ultrasonic array elements are amplified and collected by a beam former, and radio frequency (RF) data that have been transmitted by the beam former may be demodulated to obtain an amplitude envelope to thereby form an ultrasonic image. Thereafter, the amplitude envelope data are converted into synthetic image data by a scan converter and then are processed into an ultrasonic image by a video processor to be displayed in a monitor.

In the course of obtaining reception data to form an ultrasonic image, signals that are scattered and returned from small reflectors have ununiform amplitudes while being collected by an ultrasonic collection system, and thus ultrasonic images have inherent noise images called speckle. Such speckle is placed on an image of a human organ and hinders a detailed diagnosis of areas. Accordingly, it is important to remove speckle noise from an image to thereby enhance a quality of an ultrasonic image.

To remove such speckle noise, image processing uses difference in characteristics between the speckle noise and a desired image signal. In general, noise has a high frequency and thus speckle noise may be reduced from an ultrasonic image through low pass filter filtering. In such case, however, even boundaries of organs which have a high frequency are also removed from the ultrasonic image even though such boundaries should be shown with a high resolution in the image.

The foregoing prior art has such a disadvantage that characteristics of an entire image vary depending on properties of a filter used. Therefore, it is disclosed herein that an image of a target or an ultrasonic beam is formed on the basis of delay characteristics, in connection with the technology for enhancing quality of ultrasonic images or improving ultrasonic beam-forming.

The present invention offers a method for enhancing quality of ultrasonic images that is different from the methods or technologies disclosed by the prior art or other known arts. The purpose of the present invention is to reduce speckle noise and to enhance quality of images based on calculation of correlation of ultrasonic signals transmitted by respective elements of an array transducer that is made to identify whether signals of image points are those reflected by organs or are speckle noise.

### PRIOR ART LITERATURE

### Non-Patent Literature

1) M. A. Lediju, G. E. Trahey, B. C. Byram and J. J. Dahl, “Short-Lag Spatial Coherence of Backscattered Echos: Imaging Characteristics,” IEEE Transaction of Ultrasonic Ferroelectronics, and Frequency Control, vol. 58, no. 7, pp. 1377-1388, 2011.
2) P. Perona and J. Malik, “Scale-space and edge detection using anisotropic diffusion,” in Proceedings of IEEE Computer Society workshop on Computer Vision, pp. 12-27, 1989.

### SUMMARY

Accordingly, one or more exemplary embodiments provide a module for processing an ultrasonic signal based on spatial coherence and a method for processing an ultrasonic signal that is configured to obtain ultrasonic images with enhanced quality by differently filtering image forming data that has been obtained through a normal signal processing method for obtaining ultrasonic images, according to spatial correlation between reception signals transmitted by ultrasonic elements and application of weight of area characteristics.

The foregoing and/or other aspects may be achieved by providing a module for processing an ultrasonic signal that receives an ultrasonic signal reflected from the inside of a human body and forms an image based on the ultrasonic signal, the module for processing an ultrasonic signal comprising: a transducer comprising a plurality of reception elements to receive the reflected ultrasonic signal; a radio frequency (RF) signal processor configured to process signals transmitted to the transducer; a spatial coherence calculation unit configured to calculate spatial coherence from the received ultrasonic signal; a label unit configured to apply a value of an area, which is to be visualized, according to area characteristics, based on the value processed by the spatial coherence calculation unit; a filter unit configured to correct peculiarity of the label unit; and an image enhancement filter configured to enhance an image by differently applying characteristics of the filter depending on spatial coherence.

The foregoing and/or other aspects may be achieved by providing the module for processing an ultrasonic signal, wherein a type of the filter unit or the image enhancement filter or a filter value is a user-defined parameter.

The foregoing and/or other aspects may be achieved by providing a method for processing an ultrasonic signal, comprising a delay function to grant a delay time function to respective reception elements to obtain a collected signal for a specific area.

The foregoing and/or other aspects may be achieved by providing a method for processing an ultrasonic signal comprising: receiving an ultrasound that has been reflected by a diagnosed target in a human body; converting the received ultrasonic signal into radio frequency (RF) data by granting delay time to respective reception elements to obtain a collected signal for a specific area; calculating spatial coherence with respect to the RF data and giving a weight according to characteristics of the diagnosed target; filtering peculiarity of the characteristics; and enhancing an image by differently applying characteristics of a filter according to the spatial coherence.

### BRIEF DESCRIPTION OF THE DRAWINGS

The above and/or other aspects will become apparent and more readily appreciated from the following description of the exemplary embodiments, taken in conjunction with the accompanying drawings, in which:

FIG. 1 is a block diagram of an embodiment of a method for processing an ultrasonic signal according to the present invention;

FIG. 2 illustrates an embodiment of a signal processing process at each phase of the method for processing an ultrasonic signal according to the present invention;

FIG. 3 illustrates an embodiment of a spatial coherence calculation unit that may apply to the method for processing an ultrasonic signal according to the present invention;

FIG. 4 illustrates an embodiment of an ultrasonic signal processing module according to the present invention; and

FIG. 5 illustrates an embodiment of a method for processing an ultrasonic signal according to the present invention.

### DETAILED DESCRIPTION OF EXEMPLARY EMBODIMENTS

Below, exemplary embodiments will be described in detail with reference to accompanying drawings so as to be easily realized by a person having ordinary knowledge in the art. The exemplary embodiments may be embodied in various shapes without being limited to the exemplary embodiments set forth herein. Descriptions of well-known parts are omitted for clarity, and like reference numerals refer to like elements throughout.

To remove speckle noise and to enhance quality of images, an ultrasonic image for which an envelope has been detected is processed through a low pass filter by using characteristics of noise that has a high frequency. In such case, however, the part of the ultrasonic image that has a high frequency like a boundary of organs is also processed through the lower pass filter and thus speckle is reduced from the ultrasonic image but also other information is lost.

According to the present invention, the filter differently applies by using difference in characteristics of speckle noise in the area from which a signal is obtained and of a target image signal. A transmitted ultrasound is reflected by a reflector and shows different reflection characteristics depending on the size of reflectors with respect to the ultrasonic wavelength. Internal human organs are sufficiently large compared to the wavelength, and the reflected signals become signals with high correlation and in a similar shape when transmitted to ultrasonic reception elements. However, signals from the area in which reflectors are smaller than the ultrasonic wavelength are scattered and are differently incident to elements of the transducer. Accordingly, by calculating correlation of signals transmitted to the elements of the transducer, it can be identified whether such signals are from human organs or from small reflectors. In the present invention, adaptive filtering separately applies to organ areas and to speckle areas according to correlation of received signals to thereby reduce speckle and to enhance quality of images.

According to the present invention, correlation of ultrasonic signals transmitted to respective elements of an array transducer is calculated to identify whether signals from image points are those reflected by organs or are speckle noise.

According to the present invention, an ultrasonic image system processes ultrasonic images and simultaneously calculates correlation of areas from which images are to be provided and uses the calculation result in filtering and removing speckle from ultrasonic images to thereby enhance quality of images. In this specification, spatial coherence and spatial correlation are used as the same or similar meaning.

This will be described later in more detail.

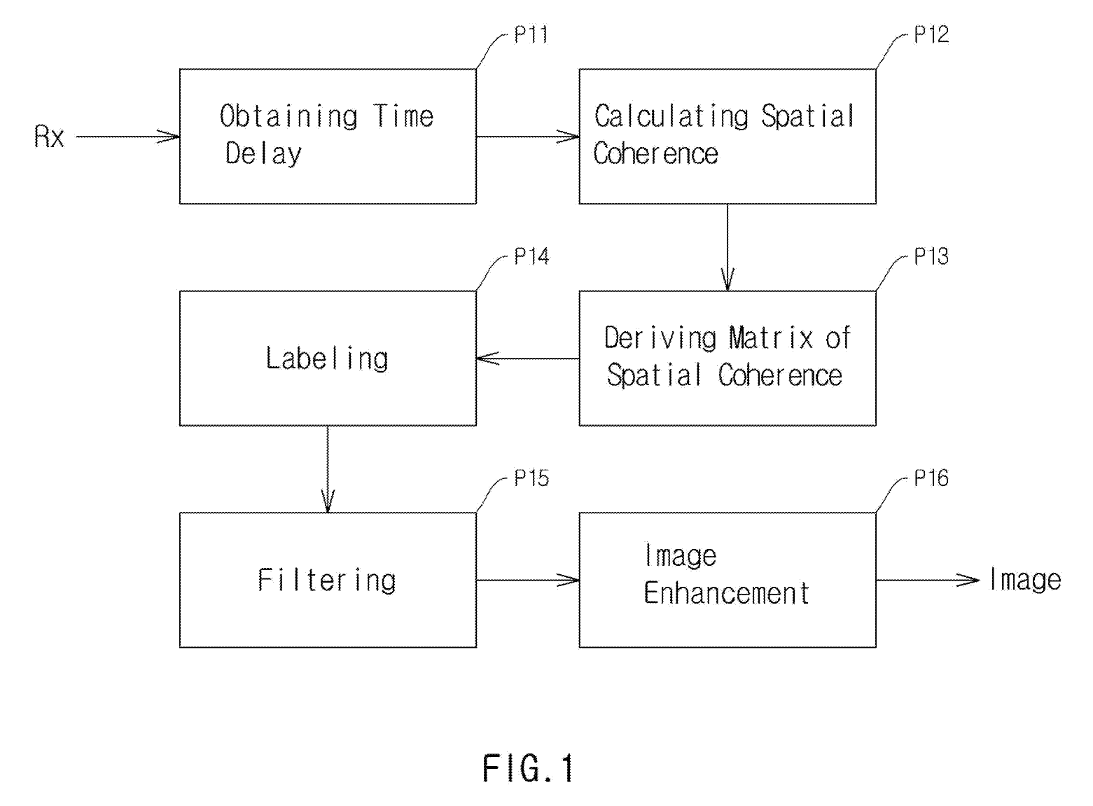

FIG. 1 is a block diagram of an embodiment of a method for processing an ultrasonic signal according to the present invention.

A process for processing an ultrasonic signal according to the present invention may include an operation of receiving by respective elements of a transducer in a time-delayed form an ultrasonic signal Rx that has been transmitted to a human body and then reflected by a predetermined area (P11); an operation of calculating spatial correlation based on the time-delayed reception ultrasonic signal received by the ultrasonic elements of the transducer (P12); an operation of forming a spatial coherence matrix from the calculated spatial correlation (P13); an operation of calculating a label matrix according to characteristics of reflected areas within a human body in addition to the spatial coherence matrix (P14); detecting and filtering peculiarity according to a peripheral area based on label result (P15); and an operation of enhancing an image by applying an adaptive filter to an image based on the filtered label result (P16).

The purpose of the method or device according to the present invention is to enhance quality of B-mode ultrasonic images, and the foregoing method is characterized by enhancing quality of a B-mode image by adding an adaptive filtering process to a calculated spatial coherence. The device or method for processing an ultrasonic signal according to the present invention may apply to form a B-mode image for ultrasonic diagnosis, but not limited thereto. For example, the device or method according to the present invention may apply to obtain an image of a treated area during a highly intensive ultrasonic treatment. The device or method according to the present invention may apply to diagnosis of various modes or treatment.

An ultrasonic signal that is transmitted to a diagnosed area in a human body may be generated by a plurality of ultrasonic elements arranged in a transducer, and may be a beam-forming ultrasonic signal to form a focus on a predetermined area or be an ultrasonic signal that does not form a focus due to non-existence of a predetermined area. The signal that is reflected by the predetermined area in the human body may be transmitted to the same ultrasonic element or to an ultrasonic element that is arranged in a different location from the location of another ultrasonic element for transmission. The ultrasonic element for reception of ultrasonic signals may be arranged in a structure known in the art, and time delay of the ultrasonic signals transmitted to the respective elements may be calculated.

Spatial correlation may be calculated according to time delay of a received ultrasonic signal (P12). Spatial correlation may be calculated on the basis of time delay received by respective ultrasonic elements arranged in the transducer. Spatial correlation between ultrasonic elements that are distant from each other by m (0 or a natural number) may be calculated by the following formula 1:

$$r ( m ) = \frac{1}{N - m} {\sum }_{i = 1}^{N - m} \frac{{\sum }_{i = {n}_{2}}^{{n}_{2}} {s}_{i} ( n ) {s}_{i + m} ( n )}{\sqrt{{\sum }_{i = {n}_{2}}^{{n}_{2}} {s}_{i}^{2} ( n ) {\sum }_{i = {n}_{3}}^{{n}_{2}} {s}_{i + m}^{2} ( n )}} \qquad < Formula 1 >$$

In the formula 1 above, r(m) is spatial correlation, N is the number of entire ultrasonic elements, N−m is the size of a kernel, and sᵢ(n) refers to data of the ith ultrasonic element that has been obtained from the depth n.

If the spatial correlation between different elements is calculated by the formula 1, spatial coherence may be also calculated, and the spatial coherence r may be the sum of the spatial correlation and M. The spatial coherence r may be represented by the following formula 2:

$$r = {\sum }_{m = 1}^{M} r ( m ) \qquad < Formula 2 >$$

In the formula 2 above, r refers to spatial correlation. A single scalar is obtained with respect to a sampling location on which a focus of the reflected area in the human body is formed, and respective sampling locations may be stored in a 2D arrangement (matrix I) (P13).

If a 2D matrix (matrix I) related to spatial coherence is calculated, label characteristics may be applied according to characteristics of the reflected area (P14). Label characteristics means reflection characteristics of an ultrasonic signal. Ultrasonic signals may have different reflection characteristics depending on density or distribution of a medium. A strong reflector that is sufficiently large compared to amplitude of ultrasound has a high spatial coherence. A small medium such as blood corpuscle that is smaller compared to amplitude of ultrasound may have a low spatial coherence due to scattering of ultrasound. Accordingly, characteristics of an area, an ultrasonic image of which is to be obtained, may be calculated on the basis of the calculated spatial coherence. More specifically, a structural area that has a strong reflector such as a bone or diaphragm has a high spatial coherence while a uniform area that has a small material such as a soft tissue or the inside of blood vessel has a low spatial coherence. Such characteristics may be shown in spatial coherence, and the label value z may be +1 in a structural area with high density, and may be −1 in a uniform area with low density, and may be between −1 and +1 in an area that is in the middle of the structural area and the uniform area, and may be calculated by the following formula 3:

$$z = \{ \begin{matrix}+ 1 , & r \geq {t}_{2} \\ - 1 , & r \leq {t}_{1} \\ \frac{r - {t}_{1}}{{t}_{2} - {t}_{1}} , & otherwise\end{matrix} \qquad < Formula 3 >$$

In the formula 3 above, t1 and t2 refer to critical values of a uniform area and a structural area.

If a label value of the area is applied, existence or non-existence of peculiarity in peripheral areas may be detected. Existence or non-existence of peculiarity may be independently detected for the uniform area and the structural area. According to the detection result, the peculiarity may be filtered and removed (P15). The peculiarity that exists in the uniform area is corrected depending on the standardization of a form. If a form has a certain size and a certain shape, it is processed as a signal to be distinguished from other peripheral areas. If a form has no certain size or has no certain shape, it may be processed as noise. The certain size or shape that acts as a basis may be determined depending on the inspected area in a human body. For example, if a known form exists in an inspected are and a similar signal has been detected, the form may be processed as an image signal. If the form has no contrast ratio that is distinguished from peripheral areas, it may be processed as noise and may be smoothed to have correlation with peripheral areas as will be described later. Peculiarity in the structural area may be basically processed as an image signal, and may be processed as a noise signal by taking into account a contrast ratio of peripheral areas.

Median filtering may apply to the peculiarity in the structural area. The median filtering may be a non-linear digital filter known in the art that is used to reduce noise. For example, a linear Gaussian filter may be used. Filtering result of the peculiarity may be represented as a 2D matrix (matrix A), and the 2D matrix is applied with an adaptive filter to enhance images together with spatial correlation matrix by the image enhancement unit.

Image enhancement may be performed to determine an image with respect to an ultrasonic signal to display the image by a display unit or to enhance quality of a displayed image. A digital signal to which the adaptive filtering has applied according to the peculiarity is finally processed and shows image enhancement (16) and may be displayed by a display unit.

Image enhancement may include an operation of smoothing peculiarity to remove noise from a uniform area and of processing peculiarity into an image signal with respect to a structural area. More specifically, the matrix A that has been obtained at the filtering operation may apply to the matrix I that represents spatial correlation to thereby correct the matrix I. For example, a diffusing filter that is represented by the following formula 4 may apply to a B-mode image matrix I:

$$\frac{\partial I}{\partial t} = div ( c ∇ I ) \qquad < Formula 4 >$$

In the formula 4 above, div refers to divergence and c refers to a diffusion coefficient. The diffusion coefficient c may be represented by the following formula 5:

$$c = f ( A ; σ ) = 1 - \frac{1}{1 + exp [ - ( A - m ) / σ ]}$$

In the formula 5 above, A refers to the matrix A that has been obtained at the filtering operation, and m may be a user-defined parameter.

If the matrix A that has area characteristics applies to the matrix I related to spatial coherence by the formula 4, speckle that amounts to noise may be reduced from, e.g., a B-mode image. Then, the signal that has an enhanced quality through adaptive filtering may be scan-converted and may be displayed by a display unit, and an enhanced image may be obtained.

The methods for calculating the spatial coherence and for calculating the label value or the filtering method are examples and the present invention is not limited to the foregoing embodiment.

Hereinafter, units that apply to the module for processing an ultrasonic signal according to the present invention will be described.

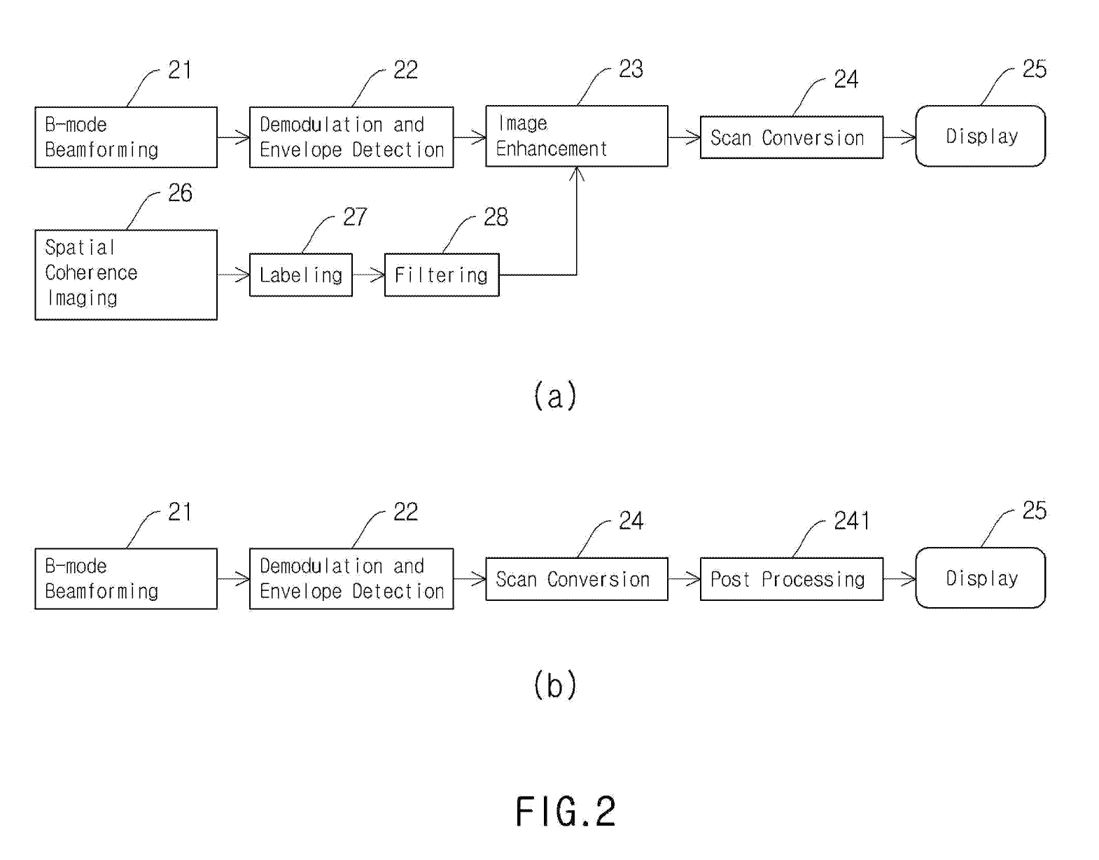

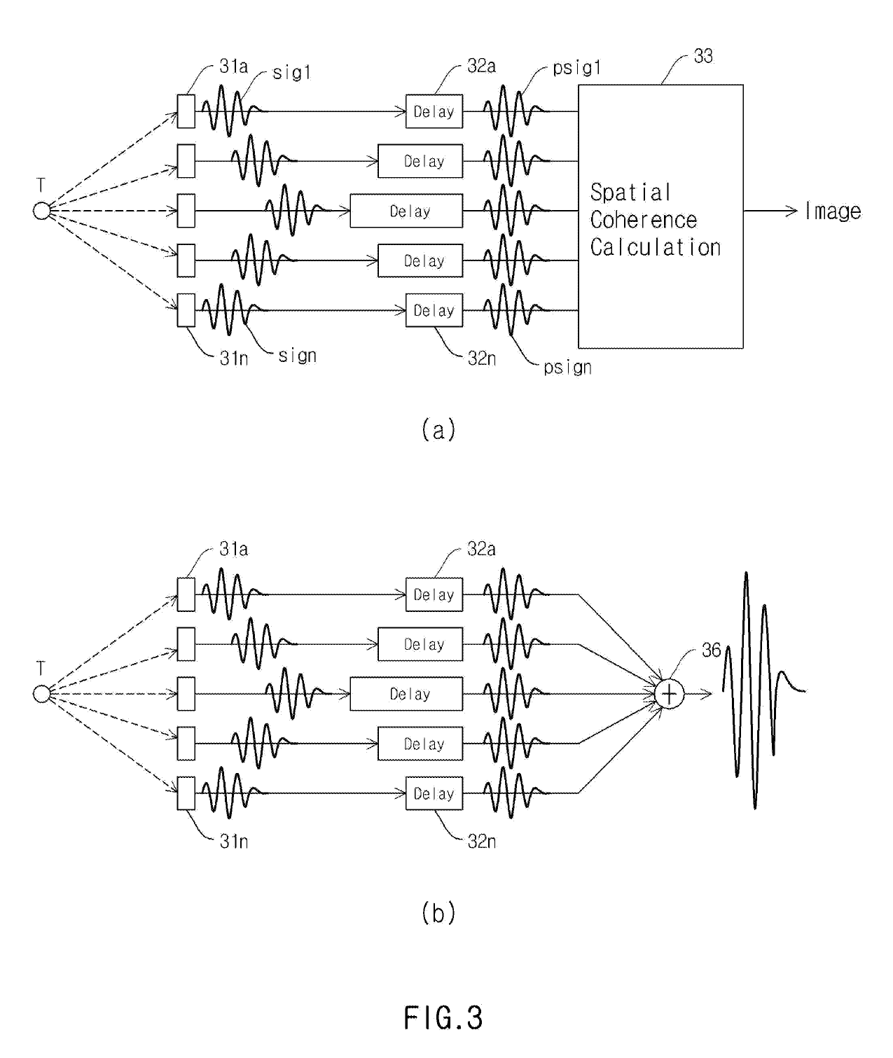

FIG. 2 illustrates an embodiment of a process of processing signals at respective operations of the method for processing an ultrasonic signal according to the present invention. FIG. 3 illustrates an embodiment of application of a spatial coherence calculation unit that may apply to the method for processing an ultrasonic signal according to the present invention.

(A) in FIG. 2 is a block diagram showing an embodiment of the process of processing signals according to the present invention. (B) in FIG. 2 illustrates an embodiment of a known signal processing process for comparison. (A) and (B) in FIG. 3 illustrate embodiments of the signal processing process according to the present invention and a known signal processing process that correspond to the beam forming and calculation of spatial coherence in (A) and (B) in FIG. 2.

Referring to (A) in FIG. 2 and FIG. 3, an ultrasonic signal processing module may include transducers 31a to 31n that include a plurality of piezoelectric elements to receive the reflected ultrasonic signal; delay signal processors 31a to 32n that apply delay for beam collection of the signal transmitted by the transducers 31a to 31n; a spatial coherence calculation unit 33 that calculates spatial coherence from the reflected ultrasonic signal; and a filter unit 28 that filters the signal processed by the delay signal processors 32a to 32n to remove peculiarity of the value that has been calculated by the spatial coherence calculation unit 33.

An ultrasonic signal that has been reflected by a diagnosed area T in a human body may be transmitted to ultrasonic elements 31a to 31n arranged in the transducers, and the signal is time-delayed from the diagnosed area T that amounts to a focus location, depending on the relative location of the respective ultrasonic elements 31a and 31b and is transmitted to the ultrasonic elements 31a to 31n. The delay time of the respective signals may be compensated for by the delay signal processors 21a to 32n. The foregoing process may be performed by a beam forming unit 21. The signals with respect to which time delay has been compensated for by the beam forming unit 21 may be demodulated by a wave form detection unit 22 and amplitude of the signals may be detected and the respective signals may be synthesized.

Referring to (B) in FIG. 2 and (B) in FIG. 3, the signals that have been processed by the delay signal processors 32a to 32n may be combined into a single signal by the wave form forming unit 36, and may be converted into a signal representing a diagnosed area through an envelope detection unit 22 and then may be transmitted to a scan converter 24. Then, the signals may be processed by a post processing processor 241, and transmitted to, and displayed as an image by, the display unit 25. The signal processing module according to the present invention may calculate spatial coherence from the signals that have been processed by the delay signal processors 32a to 32n.

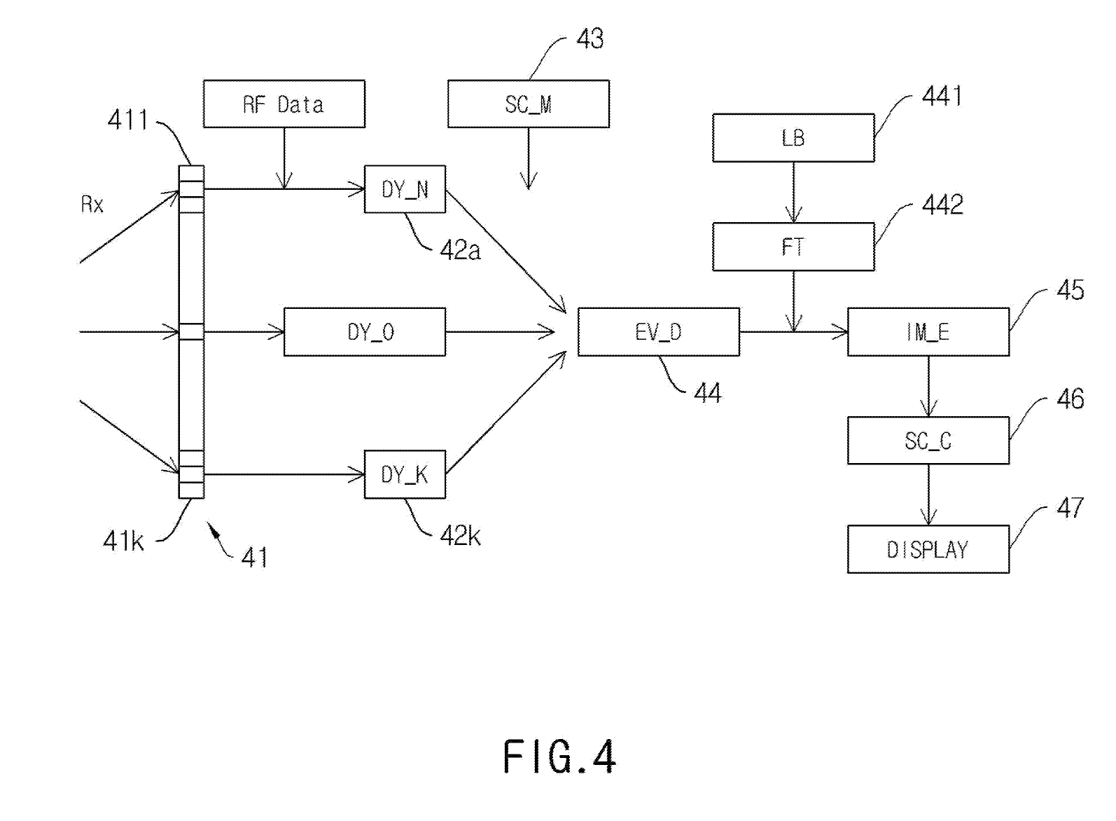

Referring to (A) in FIG. 2 and (A) in FIG. 3, the signal with respect to which time delay has been compensated for by the delay signal processors 32a to 32n is processed by the spatial coherence calculation unit 33 to be used to enhance images. More specifically, the spatial coherence calculation unit 33 may include a correlation calculation unit 26, a label unit 27 and a filter unit 28. As described above, the correlation calculation unit 26 may generate a correlation matrix I while the label unit 27 may generate an area characteristics matrix A to which a weight is given depending on the uniform area and the structural area. The matrix I and the matrix A may be transmitted to, and filtered by, the filter unit 28 to remove peculiarity of the uniform area and the structural area. For example, the filter unit 28 may include a median filter, an adaptive filter or a diffusing filter. The filter unit 28 may generate a 2D matrix as a correction value for the diagnosed area T on the respective areas and such 2D matrix may be transmitted to the image enhancement unit 23. The image enhancement unit 23 may apply the adaptive filter to the signal, to which time delay has been compensated for and wave form has been synthesized, according to the matrix provided by the filter unit 28. The finally processed signal may be transmitted to the scan converter 24 and may be displayed by the display unit 25. The foregoing process is shown in FIG. 4.

In the embodiment in FIG. 3, the label unit 27 may give a weight depending on area characteristics of the area which is to be visualized. The filter unit 28 may detect and correct peculiarity of the label unit 27. In the course of correction, characteristics of the filter may be changed on the basis of the value calculated by the spatial coherence calculation unit 33 or the correlation calculation unit 26. The value of the filter unit 28 or the image enhancement filter may be determined on the basis of the value calculated by the label unit 27 or the correlation calculation unit 26 and on characteristics of the diagnosed area. That is, the filter value may be a user-defined parameter.

FIG. 4 illustrates an embodiment of the module for processing an ultrasonic signal according to the present invention.

Referring to FIG. 4, a reflected ultrasonic signal Rx may be received by respective ultrasonic elements 411 to 411k of a transducer 41. The signal that has been received by the ultrasonic element 411 may be converted into an electric signal to become RF data, and may be transmitted to a detection unit 44 after delay time of the signal is compensated for by beam forming units 42a to 42k. The signal may be transmitted by the beam forming units 42a to 42k to the correlation unit 43 to calculate spatial correlation of the ultrasonic elements 411 to 411k depending on correlative locations thereof. The detection unit 44 may generate an envelope, and accordingly, a preliminary image signal may be formed with respect to the diagnosed area. The preliminary image signal does not practically have characteristics of the diagnosed area and thus needs to be corrected. The label unit 441 shows characteristics of the area from which a signal has been transmitted to the uniform area or the structural area, and the filter unit 442 filters and removes peculiarity. An adaptive filter may apply to the signal depending on characteristics of the uniform area and the structural area and then the signal may be transmitted to the image enhancement unit 45 for image enhancement and then to the scan converter 46 and then transmitted to and displayed as an image by the display unit 47.

In the embodiment in FIGS. 3 and 4, the correlation unit, the label unit or the filter unit are illustrated as independent elements, and also be integrated into a single processor or processing device. Likewise, the delay signal processor may be integrated into a single processor together with the correlation unit, the label unit or the filter unit. The correlation unit, the label unit or the filter unit may be hardware or software.

As described above, the module or method for processing an ultrasonic signal according to the present invention enhances quality of an ultrasonic image or a B-mode ultrasonic image through reduction of speckle resulting from area characteristics of the diagnosed area or blurring in an edge of the area rather than enhancing quality of an image through improvement of a contrast ratio of an image. As the contrast ratio of the ultrasonic image is improved, the image may be clearer.

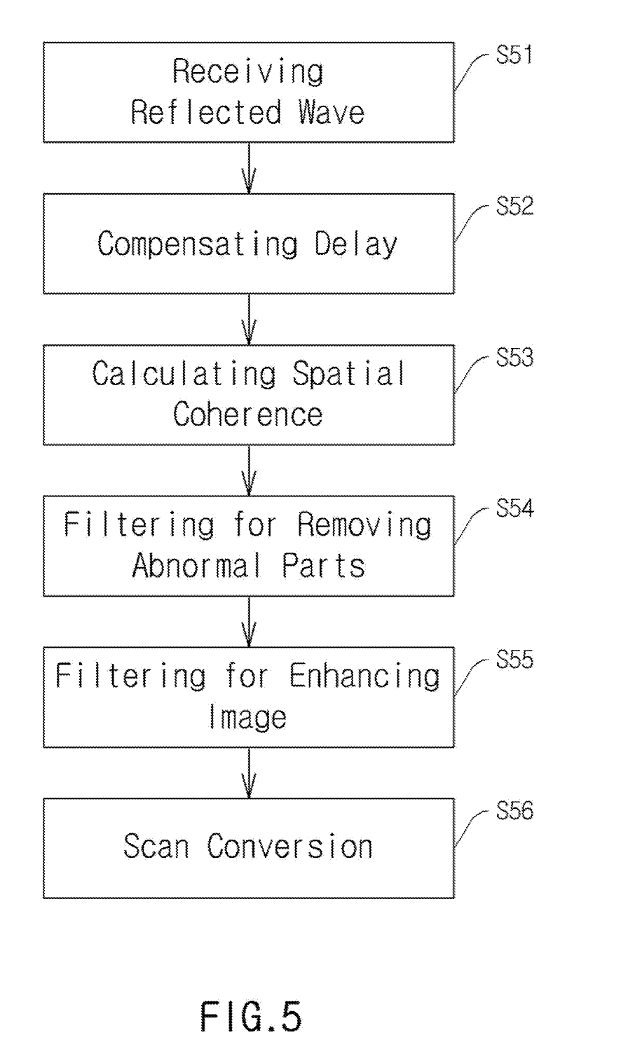

FIG. 5 illustrates an embodiment of the method for processing an ultrasonic signal according to the present invention.

Referring to FIG. 5, the method for processing an ultrasonic signal according to the present invention includes an operation of receiving a reflective wave from a diagnosed area (S51); an operation of compensating for delay with respect to respective reflected waves (S52); an operation of calculating spatial correlation from time delay data of the received reflected waves (S53); an operation filtering peculiarity from the correlation value (S54); an operation of applying different adaptive filters to the obtained correlation value depending on the uniform area or the structural area (S55); and an operation of transmitting image data that has been enhanced through the filter to the scan converter (S56).

Reflected waves may be received by respective reception elements arranged in the transducer. Time delay of the signals that have been received by the respective reception elements is compensated for and thus the signals may be converted into wireless RF data. Spatial correlation may be calculated from the converted RF data (S53). Spatial correlation may be calculated by the foregoing method, and for example, may be expressed as a matrix. As necessary, a weight may be given depending on area characteristics. Based on the spatial correlation, peculiarity may be filtered (S54). The type of the filter may be determined on the basis of the spatial correlation or according to area characteristics. The filter value may be a user-defined parameter. The image enhancement is performed by the adaptive filter (S55), and enhanced image data may be obtained. The enhanced image data may be transmitted to the scan converter and displayed as an image (S56).

In processing the ultrasonic signal according to the present invention, filtering may be performed by various methods, and the present invention is not limited to the foregoing embodiment.

The module for processing an ultrasonic signal according to the present invention defines characteristics of an area to be visualized on the basis of a value obtained from spatial coherence, and applies the characteristics to process the ultrasonic signal for image enhancement to thereby obtain an enhanced ultrasonic image. Also, the method for processing an ultrasonic signal according to the present invention reduces speckle from a B-mode image.

As described above, a module for processing an ultrasonic signal according to the present invention differently filters signals that have been obtained on the basis of spatial coherence, depending on characteristics of peripheral areas to thereby obtain an enhanced ultrasonic image. Also, a method for processing an ultrasonic signal reduces speckle from a B-mode image.

Although a few exemplary embodiments have been shown and described, it will be appreciated by those skilled in the art that changes may be made in these exemplary embodiments without departing from the principles and spirit of the invention, the range of which is defined in the appended claims and their equivalents.

What is claimed is:

1. A module for processing an ultrasonic signal that receives an ultrasonic signal reflected from the inside of a human body and forms an image based on the ultrasonic signal, the module for processing an ultrasonic signal comprising:
a transducer comprising a plurality of reception elements to receive the reflected ultrasonic signal;
a radio frequency (RF) signal processor configured to process signals transmitted to the transducer;
a spatial coherence calculation unit configured to calculate spatial coherence from the received ultrasonic signal;
a label unit configured to apply a value of an area, which is to be visualized, according to area characteristics, based on the value processed by the spatial coherence calculation unit;
a filter unit configured to correct peculiarity of the label unit; and
an image enhancement filter configured to enhance an image by differently applying characteristics of the filter depending on spatial coherence.

2. The module for processing an ultrasonic signal according to claim 1, wherein a type of the filter unit or the image enhancement filter or a filter value is a user-defined parameter.

3. The module for processing an ultrasonic signal according to claim 1, comprising a delay function to grant a delay time function to respective reception elements to obtain a collected signal for a specific area.

4. A method for processing an ultrasonic signal comprising:
receiving an ultrasound that has been reflected by a diagnosed target in a human body;
converting the received ultrasonic signal into radio frequency (RF) data by granting delay time to respective reception elements to obtain a collected signal for a specific area;
calculating spatial coherence with respect to the RF data and giving a weight according to characteristics of the diagnosed target;
filtering peculiarity of the characteristics; and
enhancing an image by differently applying characteristics of a filter according to the spatial coherence.
